(19) United States
(12) Patent Application Publication
(10) Pub. No.: US 2011/0054284 A1
(43) Pub. Date: Mar. 3, 2011

(54) Anti-Coagulant Calibrant Infusion Fluid Source

(75) Inventor: Henry W. Oviatt, Temecula, CA (US)

(73) Assignee: Edwards Lifesciences Corporation, Irvine, CA (US)

(21) Appl. No.: 12/550,151

(22) Filed: Aug. 28, 2009

(51) Int. Cl. A61B 5/1473; G01N 33/487; G01N 27/26

(52) U.S. Cl. 600/365; 204/403.01

(57) ABSTRACT

Methods and systems for preventing or eliminating thrombus during use of a sensor are disclosed. The method comprises providing a calibrant infusion fluid source comprising a predetermined amount of a calibrant and adding a predetermined amount of a non-heparin anti-thrombotic agent into the calibrant infusion fluid source. A system and method is disclosed that includes an infusion calibrant source comprising a predetermined amount of a calibrant and a predetermined amount of a non-heparin anti-thrombotic agent with a glucose sensor.

### FIELD

In general, embodiments herein disclosed relate to analyte measuring systems and, more specifically, methods and systems comprising an anticoagulant calibrant infusion fluid source for an analyte sensor.

### BACKGROUND

Controlling blood glucose levels for diabetics and other patients can be a vital component in critical care, particularly in an intensive care unit (ICU), operating room (OR), or emergency room (ER) setting where time and accuracy are essential. Presently, one of the most reliable ways to obtain a highly accurate blood glucose measurement from a patient is by a direct time-point method, which is an invasive method that involves drawing a blood sample and sending it off for laboratory analysis. This is a time-consuming method that is often incapable of producing needed results in a timely manner. Other minimally invasive methods such as subcutaneous methods involve the use of a lancet or pin to pierce the skin to obtain a small sample of blood, which is then smeared on a test strip and analyzed by a glucose meter. While these minimally invasive methods may be effective in determining trends in blood glucose concentration, they generally do not track glucose frequently enough to be practical for intensive insulin therapy, for example, where the impending onset of hypoglycemia could pose a very high risk to the patient.

Electrochemical sensors have been developed for measuring various analytes in a aqueous or physiological fluid mixture, such as the measurement of glucose in blood or serum. An analyte is a substance or chemical constituent that is determined in an analytical procedure, such as a titration. For instance, in an immunoassay, the analyte may be the ligand, antibody, DNA fragment, or other physiological marker, whereas in blood glucose testing the analyte is glucose. Electrochemical sensors comprise electrolytic cells including electrodes used to measure an analyte. Two types of electro-chemical sensors are potentiometric and amperometric sensors.

Amperometric sensors, for example, are known in the medical industry for analyzing blood chemistry. These types of sensors contain enzyme electrodes, which typically include an oxidase enzyme, such as glucose oxidase, that is immobilized within a membrane in proximity to the surface of an electrode. In the presence of blood, the membrane selectively passes an analyte of interest, e.g. glucose, to the oxidase enzyme, after which a byproduct of the enzymatic reaction is detected at the electrode. Amperometric sensors function by producing an electric current when a potential sufficient to sustain the reaction is applied between two electrodes in the presence of the reactants. For example, in the reaction of glucose and glucose oxidase, the hydrogen peroxide reaction product may be subsequently oxidized by electron transfer to an electrode. The resulting flow of electrical current in the electrode is indicative of the concentration of the analyte of interest in the media where the sensor is located. For such sensors designed for in vivo use, it may be necessary to periodically calibrate the sensor to insure proper operation and/or adjust the sensor signal to accommodate changes occurring over time including for example, environmental deterioration of the sensor enzyme, plaque or protein build up from the host's immune system, and other causes.

Intravenous blood glucose (IVBG) sensor systems typically use a heparinized saline solution containing dextrose to provide a fixed glucose concentration for sensor flush and calibration. The IVBG sensor relies on an accurate, consistent glucose concentration in the heparinized saline-filled calibrant infusion fluid source in order to calibrate the sensor.

IVBG sensor systems typically use a calibrant infusion fluid source containing a low level of heparin to prevent clotting in the tubing or in any dead-volume spaces of the sensor assembly used to sample blood for the glucose measurement from a patient. The risk of heparin-induced thrombocytopenia in human patients in addition to recent issues with contaminated heparin sources (a biological product) make the use of heparin as an anti-clotting agent a less attractive option to the medical community.

Furthermore, inadequate buffering of the infusion solution used in IVBG sensor systems could destabilize the sensor enzyme resulting in an erroneous glucose calibration reading, leading to erroneous calibration points. Such calibration errors are especially problematic for IVBG sensor measurements in a high glucose range. As a result, an alternate system of providing anti-coagulation is sought in addition to assuring stable sensor behavior during the calibration step such that reliable and stable results are obtained when the sensor is measuring analyte in blood.

### SUMMARY

The following presents a simplified summary of one or more embodiments in order to provide a basic understanding of such embodiments. This summary is not an extensive overview of all contemplated embodiments, and is intended to neither identify key or critical elements of all embodiments, nor delineate the scope of any or all embodiments. Its sole purpose is to present some concepts of one or more embodiments in a simplified form as a prelude to the more detailed description that is presented later.

In a first embodiment, a calibrant infusion fluid source is provided. The calibrant infusion fluid source comprises a container comprising a saline solution, a predetermined amount of calibrant present in the saline solution, and an effective amount of at least one non-heparin, anti-thrombotic agent present in the saline solution. The calibrant infusion source is adaptable to an intravenous glucose sensor.

In a first aspect of the first embodiment, the container is an IV bag.

In a second aspect, alone or in combination with one or more of the previous aspects of the first embodiment, the calibrant infusion fluid source further comprises a buffering system with sufficient buffering capacity that a linear glucose verses current signal is obtained across a wide range of glucose values up to 1000 mg/dL glucose. In this aspect, the calibrant infusion fluid source is used to periodically calibrate the glucose sensor by exposing it to a solution of known concentration of analyte, such that the subsequent blood analyte measurement is more accurate than a measurement obtained by a system using a heparin-containing calibrant infusion fluid source, or a calibrant source without a buffer solution. In this aspect, the calibrant infusion fluid source is used to maintain a substantially constant pH during use. In one aspect, citrate ion is functions as both the non-heparin anti-thrombotic and the buffering system.

In a third aspect, alone or in combination with one or more of the previous aspects of the first embodiment, the buffering system comprises bicarbonate ion between about 20 mM and about 100 mM such as to provide a physiological pH.

In a fourth aspect, alone or in combination with one or more of the previous aspects of the first embodiment, the buffering system comprises phosphate ion between about 0.020 M and about 0.120 M such as to provide a physiological pH.

In a fifth aspect, alone or in combination with one or more of the previous aspects of the first embodiment, the buffering system comprises at least one of citrate ion, bicarbonate ion, and phosphate ion such as to provide a physiological pH.

In a sixth aspect, alone or in combination with one or more of the previous aspects of the first embodiment, the buffering system pH of the infusion fluid source is between 6.50 and 7.6.

In a seventh aspect, alone or in combination with one or more of the previous aspects of the first embodiment, the at least one non-heparin, anti-thrombotic agent is citrate, and the buffer system is selected from at least one of phosphate or bicarbonate, wherein the calibrant fluid source has an osmolality essentially the same as human blood.

In a second embodiment, a system for sensing an analyte of interest in a subject is provided. The system comprises a calibrant infusion fluid source comprising a container comprising a saline solution, a predetermined amount of calibrant present in the saline solution, and an amount of a non-heparin anti-thrombotic agent present in the saline solution sufficient to prevent or eliminate thrombus. A glucose sensor is adapted for fluid communication with the calibrant infusion fluid source, and a controller is electrically coupled to the glucose sensor.

In a first aspect of the second embodiment, the container is an IV bag.

In a second aspect, alone or in combination with one or more of the previous aspects of the second embodiment, the system further comprises a buffering system with sufficient buffering capacity that a linear glucose verses current signal is obtained across a wide range of glucose values up to 1000 mg/dL glucose.

In a third aspect, alone or in combination with one or more of the previous aspects of the second embodiment, the buffering system comprises bicarbonate ion between about 20 mM and about 100 mM such as to provide a physiological pH.

In a fourth aspect, alone or in combination with one or more of the previous aspects of the second embodiment, the buffering system comprises phosphate ion between about 0.020 M and about 0.120 M such as to provide a physiological pH.

In a fifth aspect, alone or in combination with one or more of the previous aspects of the second embodiment, the buffering system comprises at least one of citrate ion, bicarbonate ion, and phosphate ion such as to provide a physiological pH.

In a sixth aspect, alone or in combination with one or more of the previous aspects of the second embodiment, the pH of the infusion fluid source is between 6.50 and 7.6.

In a seventh aspect, alone or in combination with one or more of the previous aspects of the second embodiment, the at least one non-heparin, anti-thrombotic agent is citrate, and the buffer system is selected from at least one of phosphate or bicarbonate, wherein the calibrant fluid source has an osmolality essentially the same as human blood.

In an eighth aspect, alone or in combination with one or more of the previous aspects of the second embodiment, the system further comprises a catheter adapted to house the glucose sensor.

In a ninth aspect, alone or in combination with the eighth aspect of the second embodiment, at least one of the surfaces of the catheter may be surface treated to reduce or eliminate thrombus.

In a tenth aspect, alone or in combination with one or more of the previous aspects of the second embodiment, the system further comprises a housing adapted to receive the glucose sensor.

In an eleventh aspect, alone or in combination with the tenth aspect of the second embodiment, at least one of the surfaces of the housing is surface treated to reduce or eliminate thrombus.

In a third embodiment, a method for preventing or eliminating thrombus during use of a sensor is provided. The method comprises providing a calibrant infusion fluid source, the calibrant infusion fluid source comprising a saline solution, a predetermined amount of calibrant present in the saline solution, and an amount of a non-heparin anti-thrombotic agent sufficient to prevent or eliminate thrombus present in the saline solution. The calibrant infusion fluid is presented to an intravenously implanted sensor, where at least a portion of the sensor is in contact with blood.

In a first aspect of the third embodiment, the container is an IV bag.

In a second aspect, alone or in combination with one or more of the previous aspects of the third embodiment, the method further comprises a buffering system with sufficient buffering capacity that a linear glucose verses current signal is obtain across a wide range of glucose values up to 1000 mg/dL glucose.

In a third aspect, alone or in combination with one or more of the previous aspects of the third embodiment, the buffering system comprises bicarbonate ion between about 20 mM and about 100 mM such as to provide a physiological pH.

In a fourth aspect, alone or in combination with one or more of the previous aspects of the third embodiment, the buffering system comprises phosphate ion between about 0.020 M and about 0.120 M such as to provide a physiological pH.

In a fifth aspect, alone or in combination with one or more of the previous aspects of the third embodiment, the buffering system comprises at least one of citrate ion, bicarbonate ion, and phosphate ion such as to provide a physiological pH.

In a sixth aspect, alone or in combination with one or more of the previous aspects of the third embodiment, the pH of the infusion fluid source is between 6.50 and 7.6.

In a seventh aspect, alone or in combination with one or more of the previous aspects of the third embodiment, the at least one non-heparin, anti-thrombotic agent is citrate, and the buffer system is selected from at least one of phosphate or bicarbonate, wherein the calibrant fluid source has an osmolality essentially the same as human blood.

In an eighth aspect, alone or in combination with one or more of the previous aspects of the third embodiment, the method further comprises providing a catheter adapted to house the glucose sensor.

In a ninth aspect, alone or in combination with the eighth aspect of the third embodiment, at least one of the surfaces of the catheter may be surface treated to reduce or eliminate thrombus.

In a tenth aspect, alone or in combination with one or more of the previous aspects of the third embodiment, the method further comprises providing a housing adapted to receive the glucose sensor.

In an eleventh aspect, alone or in combination with the tenth aspect of the third embodiment, at least one of the surfaces of the housing is surface treated to reduce or eliminate thrombus.

In a twelfth aspect, alone or in combination with one or more of the previous aspects of the third embodiment, the method further comprises maintaining a substantially constant pH environment about the glucose sensor during use

### BRIEF DESCRIPTION OF THE DRAWINGS

Having thus described embodiments of the invention in general terms, reference will now be made to the accompanying drawings, which are not necessarily drawn to scale, and wherein:

FIG. 1 is a schematic diagram of a system for blood glucose monitoring, according to an embodiment of the present invention;

FIG. 2 is a flow diagram of a method for providing a calibrant infusion fluid source to a sensor, in accordance with aspects disclosed and described herein;

FIG. 3 is a flow diagram of a method for providing a calibrant infusion fluid source to a sensor, in accordance with aspects disclosed and described herein;

FIG. 4 is a flow diagram of a method for providing a calibrant infusion fluid source to a sensor, in accordance with aspects disclosed and described herein;

FIG. 5 is a flow diagram of a method for providing a calibrant infusion fluid source to a sensor, in accordance with aspects disclosed and described herein;

FIG. 6 is a flow diagram of a method for preventing or eliminating thrombus by an intravenously positioned sensor, in accordance with aspects disclosed and described herein;

FIG. 7 is a flow diagram of a method for preventing or eliminating thrombus by an intravenously positioned sensor, in accordance with aspects disclosed and described herein;

FIG. 8 is experimental data obtained for a sensor using a calibrant infusion fluid source in accordance with aspects disclosed and described herein;

FIG. 9 is measured glucose concentration verses calculated glucose concentration obtained for a sensor using a calibrant infusion fluid source in accordance with aspects disclosed and described herein;

FIG. 10 is a graphical representation of linearity obtained for the sensor of FIG. 9, using a calibrant infusion fluid source in accordance with aspects disclosed and described herein;

FIG. 11 is a graphical representation of n linearity with error obtained for the sensor of FIG. 9, using a calibrant infusion fluid source in accordance with aspects disclosed and described herein.

### DETAILED DESCRIPTION

Embodiments of the present invention will now be described more fully hereinafter with reference to the accompanying drawings, in which some, but not all, embodiments of the invention are shown. Indeed, the invention may be embodied in many different forms and should not be construed as limited to the embodiments set forth herein; rather, these embodiments are provided so that this disclosure will satisfy applicable legal requirements. In the following description, for purposes of explanation, numerous specific details are set forth in order to provide a thorough understanding of one or more embodiments. It may be evident; however, that such embodiment(s) may be practiced without these specific details. Like numbers refer to like elements throughout.

Methods and systems are defined for preparation of calibrant infusion fluid sources. In one embodiment, a calibrant infusion fluid source for an intravenous glucose sensor that does not contain heparin and prevents or eliminates blood clotting during blood sampling and measurement is provided. This method provides an intravenous glucose sensor for use in a hospital environment, and especially for use during surgical procedures that mitigates blood clotting during use thereof. This method minimizes the potential for thrombus formation, such as from the sensor when introduced into the body and upon its contact with blood.

In an embodiment, a premixed calibrant infusion fluid source is provided that includes saline solution and an anti-thrombotic agent, optionally a buffer system comprising a predetermined concentration of at least one buffer. In such embodiments, blood clotting problems are mitigated as well as pH-related sensor deterioration during calibration and measurement sampling. Thus, a calibrant infusion fluid source comprises sufficient buffering capacity capable of providing a linear glucose verses current signal across a wide range of glucose values up to and including about 1000 mg/dL glucose. This premixed calibrant infusion fluid source provides for accurate and consistent blood glucose concentration measurements during use of a intravenous glucose sensor.

It is generally believed that by providing the buffering capacity in a calibrant infusion fluid source, the signal of a glucose sensor is stabilized to an extent greater than that of a similar sensor exposed to an un-buffered infusion fluid source. While not to held to any particular theory, it is believed that the buffered calibrant infusion fluid source prevents or eliminates buildup of acidic byproduct and prevents or eliminates an acidic pH shift in and around the sensor environment by rapidly neutralizing the acidic by-products. For example, in an enzymatic glucose sensor, the gluconic acid formed in the glucose oxidase (GOx) catalyzed oxidation of glucose may be effectively neutralized, or the local environmental pH may be maintained near a predetermined value or range.

According to a first embodiment, a calibrant infusion fluid source with an anti-coagulant, such as citrate or citric acid/citrate that comprises a quantity of either phosphate or bicarbonate, either present in higher than physiological or normal concentrations but the resultant fluid having a similar osmolality to human blood, such that a stable glucose signal is provided. Citrate concentration may be between 0.5-4% wt/v % (0.019 M-0.15 M). Citric acid/citrate solutions of between about 1:2 and 1:20 molar ration (citric/citrate) may be used. Citrate may be used for providing both anti-thrombotic function as well as buffering. Citrate may be the anti-thrombotic agent and the sole component of the buffering system.

Phosphate concentration may be between about 0.020 M and about 0.120 M. Phosphate and citrate buffering systems may be comprised of between about 0.020 M and about 0.120 M phosphate and between about 0.019 M and about 0.15 M citrate.

Bicarbonate concentration may be between about 20 mM and about 100 mM such as to provide a physiological pH. Bicarbonate and citrate buffering systems may be comprised of between about 20 mM and about 100 mM bicarbonate and between about 0.019 M and about 0.15 M citrate. As used herein, “bicarbonate” or “bicarbonate ion” is inclusive of carbonate ions and the mixture of bicarbonate and carbonate ions normally or abnormally present in biological fluids.

Phosphate/bicarbonate/citrate buffering systems concentrations may be comprised of between about 0.020 M and about 0.120 M phosphate, between about 20 mM and about 100 mM bicarbonate, and between about 0.019 M and about 0.15 M citrate. Such buffering systems can be provided in the above specified ranges provided the osmolality of the solution is not excessive (e.g., about 320 mOsm+/−10%). Sodium, potassium, and ammonium salts of citrate, bicarbonate, or phosphate may be used.

According to an aspect of the first embodiment, the calibrant infusion fluid source provides buffering capacity to an implanted intravenous blood glucose sensor such that a physiological mammalian pH range, or a pH range between a pH of about 6.50 and about 7.6, is provided.

According to other aspect of the first embodiments, the calibrant infusion fluid source comprises an anti-thrombotic agent to prevent and/or eliminate thrombus (blood clotting) in the sensor assembly during use. Anti-thrombotic agents include, for example, anti-platelet agents, thrombolytic agents, and non-heparin anticoagulants such as direct thrombin inhibitors. Suitable anti-platelet agents include P2Y12 receptor inhibitors. Suitable anti-platelet agents include thienopyridine compounds, for example, Clopidogrel, (marketed under the tradename Plavix, Clopilet, or Ceruvin), ticlopidine or prasugrel. Suitable anti-platelet agents include platelet aggregation inhibitors. Suitable thrombolytic agents include, for example, vitamin K antagonists, tissue plasminogen activators (t-PA), Alteplase (Activase), reteplase (Retavase), tenecteplase (TNKase), Anistreplase (Eminase), streptokinase (Kabikinase, Streptase), and urokinase (Abbokinase). Suitable non-heparin anticoagulants include, for example, direct throbin inhibitors or bivalent) for example, univalent direct throbin inhibitors such as Argatroban, Dabigatran, Melagatran, and Ximelagatran, or bivalent direct throbin inhibitors such as Hirudin, Bivalirudin (Angiomax), Lepirudin, and Desirudin. Other thrombotic agents may be used, such as Dabigatran, Defibrotide, Dermatan sulfate, Fondaparinux (Arixtra), and Rivaroxaban (Xarelto). Combinations of thrombotic agents as listed above may be used.

In another aspect of the first embodiment, the method provides for the calibrant infusion fluid source further including providing the calibrant infusion fluid source that includes the saline solution, the predetermined concentration of glucose and a non-heparin based anti-thrombotic agent.

In a second embodiment, a system comprising a calibrant infusion fluid source comprising an anti-thrombotic agent in combination with an intravenous glucose sensor is provided. The system includes a calibrant infusion fluid source including a saline solution, an anti-thrombotic agent, and known glucose concentration. The system additionally includes a sensor.

In one specific embodiment of the system, the calibrant infusion fluid source further includes a buffer system.

According to specific embodiments of the system, the calibrant infusion fluid source further includes the saline solution, the predetermined concentration of glucose and a non-heparin based anti-thrombotic agent.

To the accomplishment of the foregoing and related ends, the one or more embodiments comprise the features hereinafter fully described and particularly pointed out in the claims. The following description and the annexed drawings set forth in detail certain illustrative features of the one or more embodiments. These features are indicative, however, of but a few of the various ways in which the principles of various embodiments may be employed, and this description is intended to include all such embodiments and their equivalents.

The term “calibrant” as used herein is inclusive of one or more analytes of interest believed to be present in the environment of the sensor during use, and exogenous compounds or compositions of matter that may be used to calibrate a sensor. In a particularly preferred embodiment, the calibrant is glucose, glucose in combination with one or more analytes of interest other than glucose, exogenous compounds or compositions of matter that may be used to calibrate a sensor, or combinations thereof.

The method herein disclosed provides a highly accurate and convenient manner for use in a hospital environment. In one aspect, discussed in detail infra, a premixed calibrant infusion fluid source is provided that includes saline solution and a predetermined concentration of glucose together with an anti-thrombotic agent. Likewise, the phrase “glucose sensor” is inclusive of additional analyte sensors or sensors in addition to the glucose sensor.

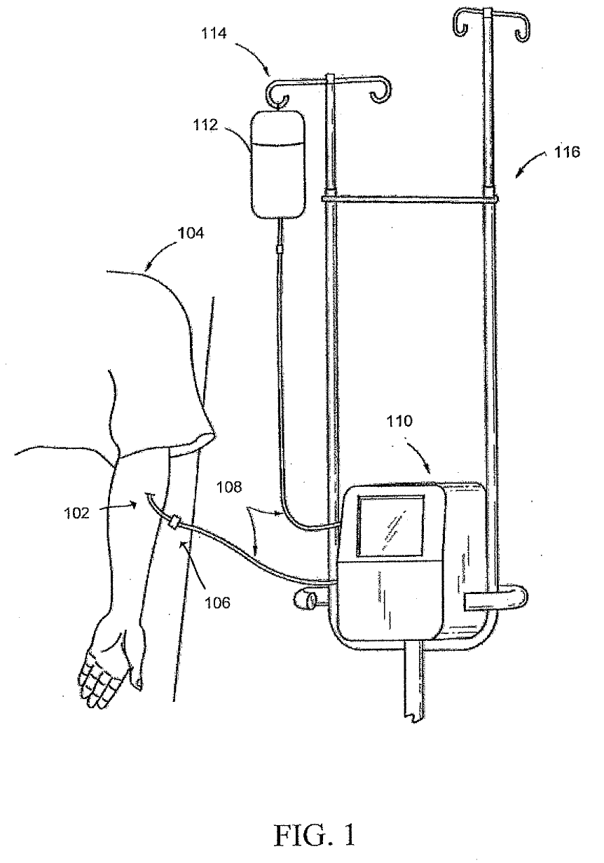

In one aspect of the present invention, the intravenous blood glucose (IVBG) sensor system illustrated in FIG. 1 is employed. System 100 of FIG. 1 includes a sensor assembly 102, for example, as described in United States Patent Application Publication No.: 2008/00860427, which is incorporated herein by reference, that is intravenously inserted to a patient 104. The sensor assembly 102 is connected to the patient via an intravenous (IV) housing 106 and an infusion line 108, which is operably connected to a fluid controller (not shown) that is controlled by a control unit 110. The housing and/or catheter may be surface treated to prevent or eliminate thrombus. Finally, the infusion line 108 continues upstream of the fluid controller to a calibrant infusion fluid source 112, such as a calibrant infusion fluid bag, which may be supported by member 114. The system may be attached to a support structure 116. In one embodiment, member 114 may serve as a scale (piezoelectric or spring) operable to weigh the bag and send the weight to the controller.

During calibration mode of system 100, control unit 110 controls and meters calibrant infusion fluid from the calibrant infusion fluid source 112, past sensor assembly 102, and into the patient 104. The sensor assemblies preferably include sensing electrodes constructed, for example, as described in U.S. Patent Application Publication Nos.: 2009/0143658, 2009/0024015, 2008/0029390, 20070202672, 2007/0202562, and 2007/0200254, which are incorporated herein by reference, and during calibration, the current generated by the respective electrodes of the sensor (e.g., a working electrode and a blank electrode) assembly is measured to provide calibration measurements for system 100.

During measurement mode of the system, blood is urged past the sensor by reversing the fluid controller. In one aspect, blood may be prevented from being withdrawn from the patient 104. In another aspect, blood from the patient may be drawn past sensor assembly 102 but preferably not past control unit 110. While blood is in contact with the sensor assembly the current or other detectable signal generated by the respective electrodes is measured.

In one embodiment, substantially the same flow rates are used during calibration mode and during measurement mode. More particularly, the control system controls the infusion of the system such that the calibrant infusion fluid is urged past the sensor electrodes at a fixed flow rate during calibration, and the blood measurement is taken while the blood is drawn back from the patient at approximately the same flow rate. Other flow rates for the calibration and measurement modes may be used.

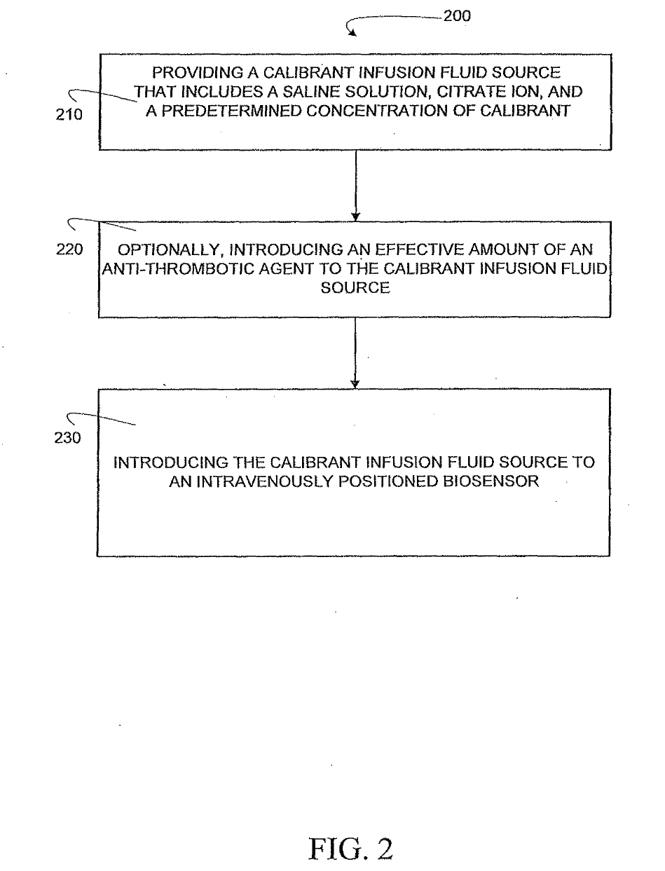

Referring to FIG. 2, a flow diagram is presented of a method 200 for preparing a calibrant infusion fluid source, in accordance with embodiments of the present invention comprising a citrate buffer. At Event 210, a predetermined concentration of calibrant (e.g., glucose) is introduced to a calibrant infusion fluid source that includes saline solution and citrate ion as buffer. The amount of predetermined quantity of glucose that is added to the calibrant infusion fluid source is proportional to the predetermined concentrate of the glucose. Thus, if higher concentrate glucose is used, a smaller volume of glucose is added and if a lower concentrate glucose is used, a larger volume of glucose is added. According to certain embodiments of the invention, as described infra., adding/injecting lower concentration glucose in higher volume provides for greater overall reliability than adding/injecting higher concentration glucose in lower volume. In one specific embodiment of the invention, 5% (by weight) dextrose injections are used as the predetermined glucose concentrate, however, it should be noted that concentrations up to and exceeding 50% dextrose/glucose may also be used. In one embodiment, in which the calibrant infusion fluid sources contain 500 mL of saline and heparin solution, the 5% dextrose-injections are of 24 mL in volume.

At Event 220, an effective amount of an anti-thrombotic agent is optionally introduced to the calibrant infusion source. The introduction of the citrate ion, the optional anti-thrombotic agent, and the predetermined concentration of calibrant may be carried out in any order or may be introduced simultaneously.

At Event 230, the calibrant infusion source comprising the citrate ion and the predetermined concentration of calibrant is introduced to an intravenously positioned sensor, e.g. a glucose sensor, thereby insuring the accuracy of the resulting concentration of glucose determined by the sensor.

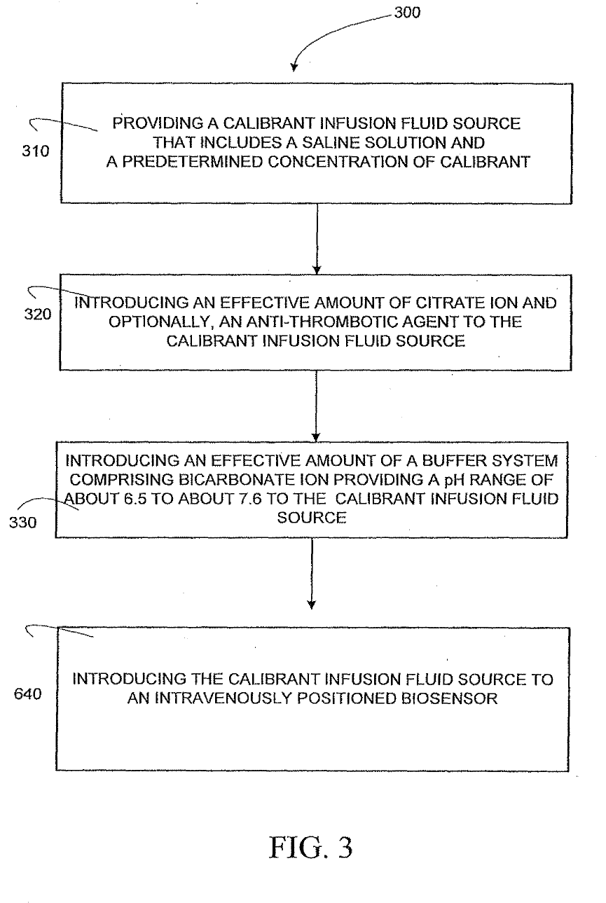

Referring to FIG. 3 a flow diagram is presented of an alternate method 300 for preparing a calibrant infusion fluid source in accordance with embodiments of the present invention comprising a source of citrate ion in combination with a bicarbonate buffer. At Event 310, a calibrant infusion fluid source is provided that includes a saline solution and a predetermined concentration of calibrant, e.g., glucose.

At Event 320, an effective amount of citrate ion and optionally an anti-thrombotic agent is introduced to the calibrant infusion source. The introduction of the citrate ion, the optional anti-thrombotic agent, and the predetermined concentration of calibrant may be carried out in any order or may be introduced simultaneously.

At Event 330, an effective amount of a buffer system comprising bicarbonate ion is introduced to the calibrant infusion source to provide a pH range of about 6.5 to about 7.6. The introduction of the bicarbonate buffer, citrate ion, and the predetermined concentration of calibrant may be carried out in any order or may be introduced simultaneously provided that a pH range of about 6.5 to about 7.6 is provided.

At Event 340, the calibrant infusion source comprising the effective amount of buffer system comprising bicarbonate ion, the effective amount of citrate ion, and the predetermined concentration of calibrant is introduced to an intravenously positioned sensor, e.g. a glucose sensor, thereby insuring the accuracy of the resulting concentration of glucose determined by the sensor.

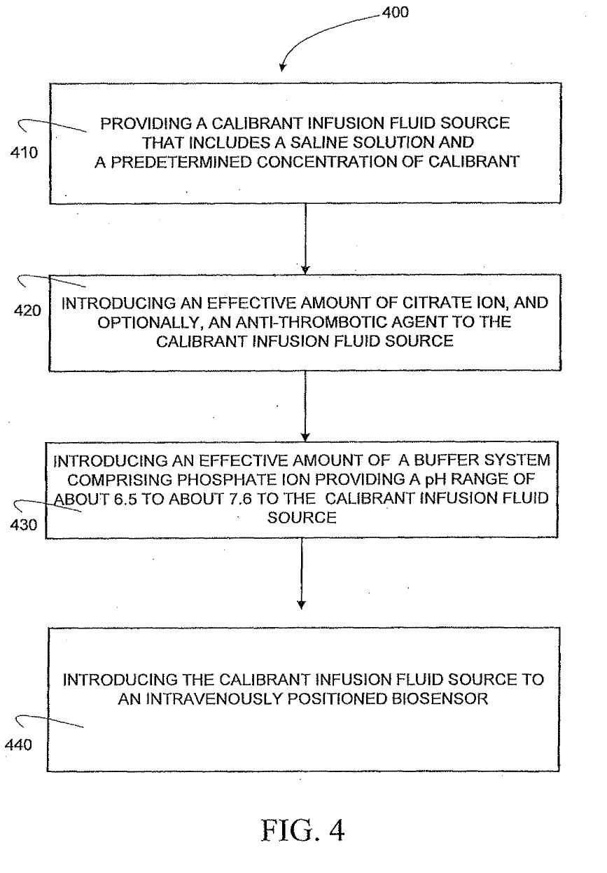

Referring to FIG. 4., a flow diagram is presented of an alternate method 400 for preparing a calibrant infusion fluid source in accordance with embodiments of the present invention comprising a source of citrate ion in combination with a bicarbonate buffer. At Event 410, a calibrant infusion fluid source is provided that includes a saline solution and a predetermined concentration of calibrant, e.g., glucose.

At Event 420, an effective amount of citrate ion and optionally an anti-thrombotic agent is introduced to the calibrant infusion source. The introduction of the citrate ion, the optional anti-thrombotic agent, and the predetermined concentration of calibrant may be carried out in any order or may be introduced simultaneously.

At Event 430, an effective amount of a buffer system comprising phosphate is introduced to the calibrant infusion source to provide a pH range of about 6.5 to about 7.6. The introduction of the phosphate buffer, citrate ion, and the predetermined concentration of calibrant may be carried out in any order or may be introduced simultaneously provided that a pH range of about 6.5 to about 7.6 is provided.

At Event 440, the calibrant infusion source comprising the effective amount of buffer system comprising phosphate, the effective amount of citrate ion, optional anti-thrombotic, and the predetermined concentration of calibrant is introduced to an intravenously positioned sensor, e.g. a glucose sensor, thereby insuring the accuracy of the resulting concentration of glucose determined by the sensor.

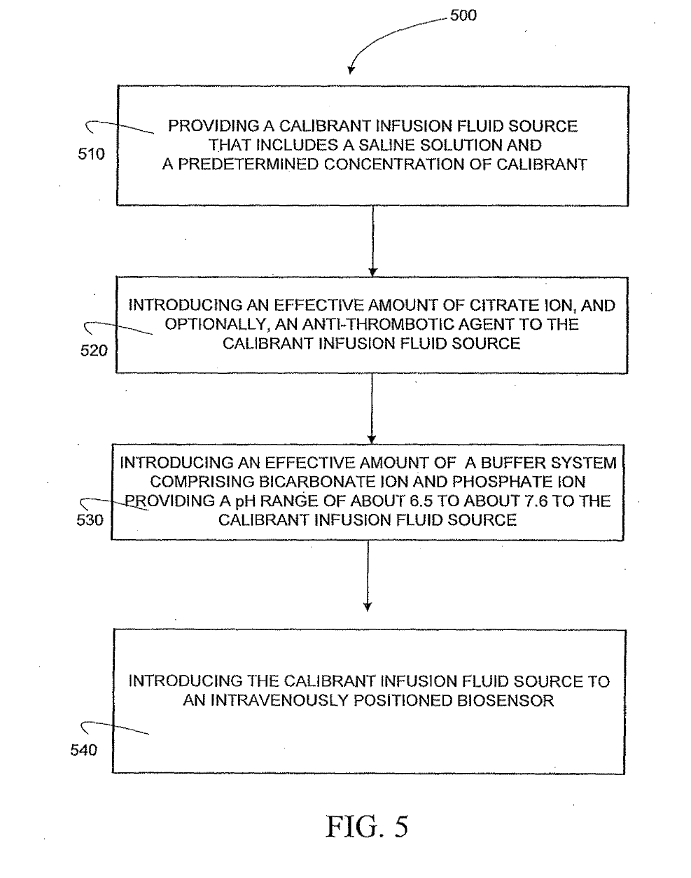

Referring to FIG. 5., a flow diagram is presented of an alternate method 500 for preparing a calibrant infusion fluid source in accordance with embodiments of the present invention comprising a source of citrate ion in combination with a bicarbonate buffer. At Event 510, a calibrant infusion fluid source is provided that includes a saline solution and a predetermined concentration of calibrant, e.g., glucose.

At Event 520, an effective amount of citrate ion and optionally an anti-thrombotic agent is introduced to the calibrant infusion source. The introduction of the citrate ion, the optional anti-thrombotic agent, and the predetermined concentration of calibrant may be carried out in any order or may be introduced simultaneously.

At Event 530, an effective amount of a buffer system comprising bicarbonate ion and phosphate is introduced to the calibrant infusion source to provide a pH range of about 6.5 to about 7.6. The introduction of the bicarbonate/phosphate buffer, citrate ion, optional anti-thrombotic agent, and the predetermined concentration of calibrant may be carried out in any order or may be introduced simultaneously provided that a pH range of about 6.5 to about 7.6 is provided.

At Event 540, the calibrant infusion source comprising the effective amount of buffer system comprising bicarbonate/phosphate, the effective amount of citrate ion, optional anti-thrombotic agent, and the predetermined concentration of calibrant is introduced to an intravenously positioned sensor, e.g. a glucose sensor, thereby insuring the accuracy of the resulting concentration of glucose determined by the sensor.

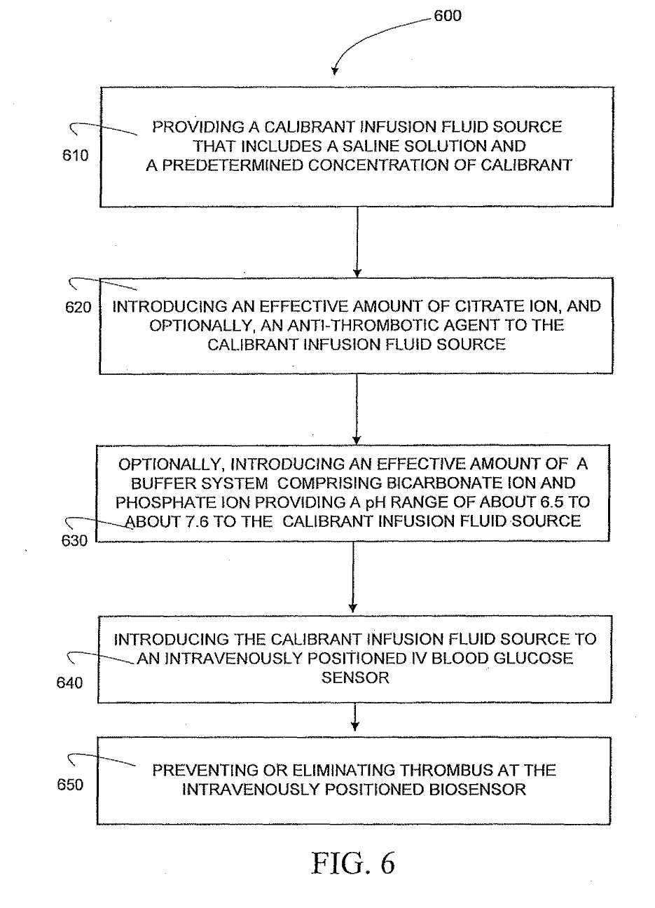

Referring to FIG. 6., a flow diagram is presented of a method 600 for preventing or eliminating thrombus in the intravenously positioned IV sensor, e.g., intravenous blood glucose sensor. At Event 610, a calibrant infusion fluid source is provided that includes a saline solution and a predetermined concentration of calibrant, e.g., glucose.

At Event 620, an effective amount of citrate ion or anti-thrombotic agent is introduced to the calibrant infusion source. The introduction of citrate ion or the anti-thrombotic agent and the predetermined concentration of calibrant may be carried out in any order or may be introduced simultaneously.

At Event 630, an effective amount of a buffer system comprising bicarbonate ion and phosphate is introduced to the calibrant infusion source to provide a pH range of about 6.5 to about 7.6. The introduction of the effective amount of citrate or anti-thrombotic agent, buffer system, and the predetermined concentration of calibrant may be carried out in any order or may be introduced simultaneously provided that a pH range of about 6.5 to about 7.6 is provided.

At Event 640, the calibrant infusion source comprising the effective amount of buffer system, the effective amount of citrate or anti-thrombotic agent, and the predetermined concentration of calibrant is introduced to an intravenously positioned sensor, e.g. a glucose sensor, preventing or eliminating thrombus therein.

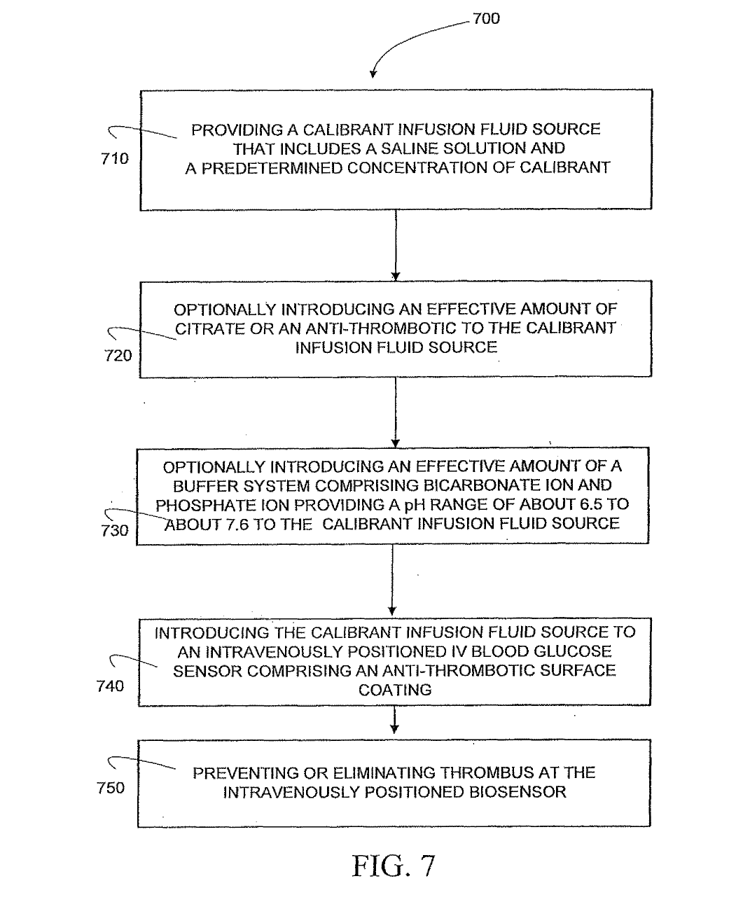

Referring to FIG. 7., a flow diagram is presented of a method 700 for preventing or eliminating thrombus in the intravenously positioned IV sensor, e.g., intravenous blood glucose sensor. At Event 710, a calibrant infusion fluid source is provided that includes a saline solution and a predetermined concentration of calibrant, e.g., glucose.

At optional Event 720, an effective amount of citrate and/or an anti-thrombotic agent is introduced to the calibrant infusion source. The introduction of citrate and/or the anti-thrombotic agent and the predetermined concentration of calibrant may be carried out in any order or may be introduced simultaneously.

At optional Event 730, an effective amount of a buffer system comprising bicarbonate ion and phosphate is introduced to the calibrant infusion source to provide a pH range of about 6.5 to about 7.6. The introduction of the effective amount of citrate and/or the anti-thrombotic agent, buffer system, and the predetermined concentration of calibrant may be carried out in any order or may be introduced simultaneously provided that a pH range of about 6.5 to about 7.6 is provided.

At Event 740, the calibrant infusion source comprising the optional effective amount of citrate and/or the anti-thrombotic agent, the optional effective amount of buffer system, and the predetermined concentration of calibrant is introduced to an intravenously positioned sensor e.g. a glucose sensor, comprising an anti-thrombotic surface coating as further described and disclosed herein. Any of the surfaces that may come into contact with blood can be surface treated, such as tubing, catheter, sensor substrate, housing, or combinations thereof.

At Event 750, the anti-thrombotic surface coated intravenously positioned sensor prevents or eliminates thrombus therein.

### Surface Coatings

Various methods may be used, alone or in combination with the infusion fluid source described above, for providing a material with a modified surface resistant to thrombus and/or having anti-thrombotic properties. For example, a sensor housing or support (e.g., catheter) may be chemically bonded to a quaternary ammonium salt and then coupled with an anti-thrombotic agent. This may be done by incorporating an amine in the polymer, quaternizing the amine, and then coupling the agent to the quaternized material to provide an ionically bound anti-thrombotic agent. Various chemical surface modifications of the sensor or support may be used to anchor the agent, for example, gas-discharge plasma methods, corona discharge surface activation, ebeam or gamma surface activation.

### Examples

Assays were conducted with a flex circuit sensor as previously described, for example, in U.S. Patent Application Publication No.: 20090143658, using a silicone catheter with a drip calibration method. Multiple points of each glucose value with a small differences between each ramp step of glucose was used. A PBS solution with 2% trisodium citrate at a calibration value of approximately 200 mg/dL glucose with a pH already adjusted to 7.4 was used. Glucose solutions comprised 0 mg/dL, 50 mg/dL, 100 mg/dL, 150 mg/dL, 200 mg/dL, 250 mg/dL, 300 mg/dL, 350 mg/dL and 400 mg/dL glucose.

Run-in for the sensors was completed with a static solution using a citrate IV bag spiked with glucose as described above. After run-in, the silicon tube was switched between the calibration drip and the glucose control solutions. Instead of switching from one glucose solution to another, a calibration drip was used in between glucose solutions and allowed to drip through the tube into a waste container. During a glucose solution introduction, the iVEK pump was used to clear the previous solution in the tubing using the “Prime” function. Then, the pump slowly withdrew the solution over a predetermined period of time using the “Dispense” function. After changing glucose solutions, the “prime” function was used to withdraw solution at 50 μL/s for the full cycle. Immediately following that, the “dispense” function was used to slowly withdraw solution at 1.5 μL/s, which took approximately 66 seconds. Two “dispense” cycles were completed before letting the calibration solution drip through the tube before switching to the next solution.

Experiment 1: Run-in was carried out at −0.85V for 10 minutes, followed by switched 0.7V. The calibration solution was 192.5 mg/dL glucose and the solution was static inside the silicone-tubing. The solution is static inside the silicone tube and placed outside (at room temperature).

After the run-in, the sensor and tubing was switched to the 50 mg/dL calibration solution and primed the sensor at a rate of 50 μL/s for the full cycle. Then, the solution was withdrawn from the solution at a rate of 1.5 μL/s. This was repeated for the remaining glucose solutions.

| | Average | | | Calculated | Error | % Error |
|---|---|---|---|---|---|---|
| [Glucose] | Signal | Calculated | Calculated | Values | (mg/dL) | ((Act − Theory)/ |
| (mg/dL) | (nA) | Slope | Intercept | (mg/dL) | (Act − Theory) | Theory) |
| 192.5 | 2.58 | 87.29 | −32.55 | | | |
| 50.75 | 0.95 | | | 50.75 | 0.00 | 0.00 |
| 192.5 | 2.86 | 81.17 | −39.37 | | | |
| 106.5 | 1.80 | | | 109.03 | −2.53 | −2.32 |
| 192.5 | 2.92 | 121.22 | −161.92 | | | |
| 157.5 | 2.64 | | | 170.28 | −12.78 | −7.50 |
| 192.5 | 3.01 | 28.54 | 106.71 | | | |
| 201.5 | 3.32 | | | 216.11 | −14.61 | −6.76 |
| 192.5 | 3.05 | 61.19 | 5.57 | | | |
| 264 | 4.22 | | | 278.58 | −14.58 | −5.23 |
| 192.5 | 3.07 | 65.62 | −9.07 | | | |
| 310.5 | 4.87 | | | 324.24 | −13.74 | −4.24 |
| 192.5 | 3.06 | 65.59 | −7.99 | | | |
| 340.5 | 5.31 | | | 358.63 | −18.13 | −5.05 |
| 192.5 | 3.08 | 65.64 | −9.36 | | | |
| 402.5 | 6.27 | | | 426.63 | −24.13 | −5.66 |
| 192.5 | 3.12 | 65.34 | −11.05 | | | |
| 402.5 | 6.33 | | | 424.68 | −22.18 | −5.22 |
| 192.5 | 3.11 | 65.60 | −11.26 | | | |
| 340.5 | 5.36 | | | 355.96 | −15.46 | −4.34 |
| 192.5 | 3.12 | 62.29 | −2.12 | | | |
| 310.5 | 5.02 | | | 328.94 | −18.44 | −5.61 |
| 192.5 | 3.08 | 60.04 | 7.48 | | | |
| 264 | 4.27 | | | 279.47 | −15.47 | −5.53 |
| 192.5 | 3.06 | 32.61 | 92.58 | | | |
| 201.5 | 3.34 | | | 212.77 | −11.27 | −5.30 |
| 192.5 | 3.05 | 103.71 | −124.09 | | | |
| 157.5 | 2.72 | | | 167.62 | −10.12 | −6.04 |
| 192.5 | 3.07 | 75.33 | −38.90 | | | |
| 106.5 | 1.93 | | | 108.86 | −2.36 | −2.17 |
| 192.5 | 3.10 | 69.40 | −22.44 | | | |
| 50.75 | 1.05 | | | 44.08 | 6.67 | 15.12 |
| 192.5 | 3.13 | 69.59 | −25.13 | | | |
| 50.75 | 1.09 | | | 45.92 | 4.83 | 10.52 |
| 192.5 | 3.14 | 75.73 | −44.96 | | | |
| 106.5 | 2.00 | | | 111.00 | −4.50 | −4.05 |
| 192.5 | 3.14 | 103.06 | −130.81 | | | |
| 157.5 | 2.80 | | | 168.14 | −10.64 | −6.33 |
| 192.5 | 3.15 | 32.93 | 88.89 | | | |
| 201.5 | 3.42 | | | 212.05 | −10.55 | −4.97 |
| 192.5 | 3.16 | 57.79 | 10.07 | | | |
| 264 | 4.39 | | | 280.70 | −16.70 | −5.95 |
| 192.5 | 3.18 | 59.95 | 1.85 | | | |
| 310.5 | 5.15 | | | 331.79 | −21.29 | −6.42 |
| 192.5 | 3.19 | 61.72 | −4.20 | | | |
| 340.5 | 5.58 | | | 361.83 | −21.33 | −5.89 |
| 192.5 | 3.19 | 63.97 | −11.86 | | | |
| 402.5 | 6.48 | | | 423.76 | −21.26 | −5.02 |
| 192.5 | 3.19 | | | | | |

Table 1 presents averaged data for each step at 2.5 minutes. Actual YSI values for the glucose concentrations were used. The repetition of the 192.5 mg/dL glucose in citrate solution was used as a re-occurring calibration point. For these calculations, the slope and intercept for every calibration point was determined and the and the glucose concentration was determined immediately following. The point at which the slope stabilized was chosen as a set point (87.29 for slope and −32.55 for intercept). After choosing the set point, the corresponding y-intercept (−32.55) became the intercept for all subsequent calibration points. Using the equation y=mx+b, each slope was recalculated with the set's intercept, “b”, (e.g., b=−32.55), and “y” (e.g., y=192.5), and solving for x providing the signal at that calibration point. From there, each signal obtained at the variable glucose level was calculated using the above equation to yield a corresponding theoretical glucose value that generally fell on the line created by the set point intercept and calibration immediately before the glucose level determination and is listed in the “Calculated Values” column. The error is the difference between the actually measured glucose value and the calculated theoretical value.

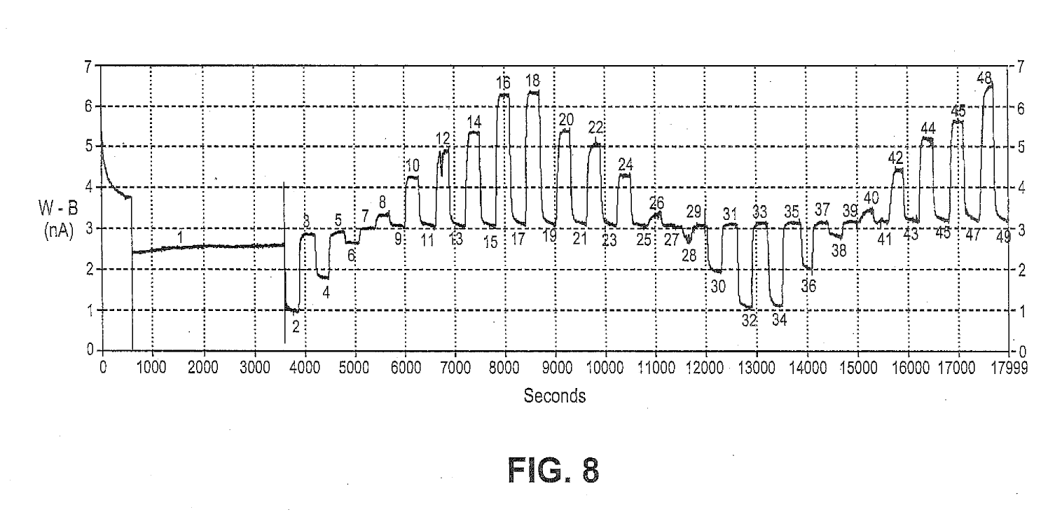

The raw data signal was taken for 30 seconds in the middle of withdrawing the solution for each step and is shown in FIG. 8 for one representative run through the glucose solutions, where the y-axis is the working electrode minus blank electrode current (nA) and the x-axis is seconds.

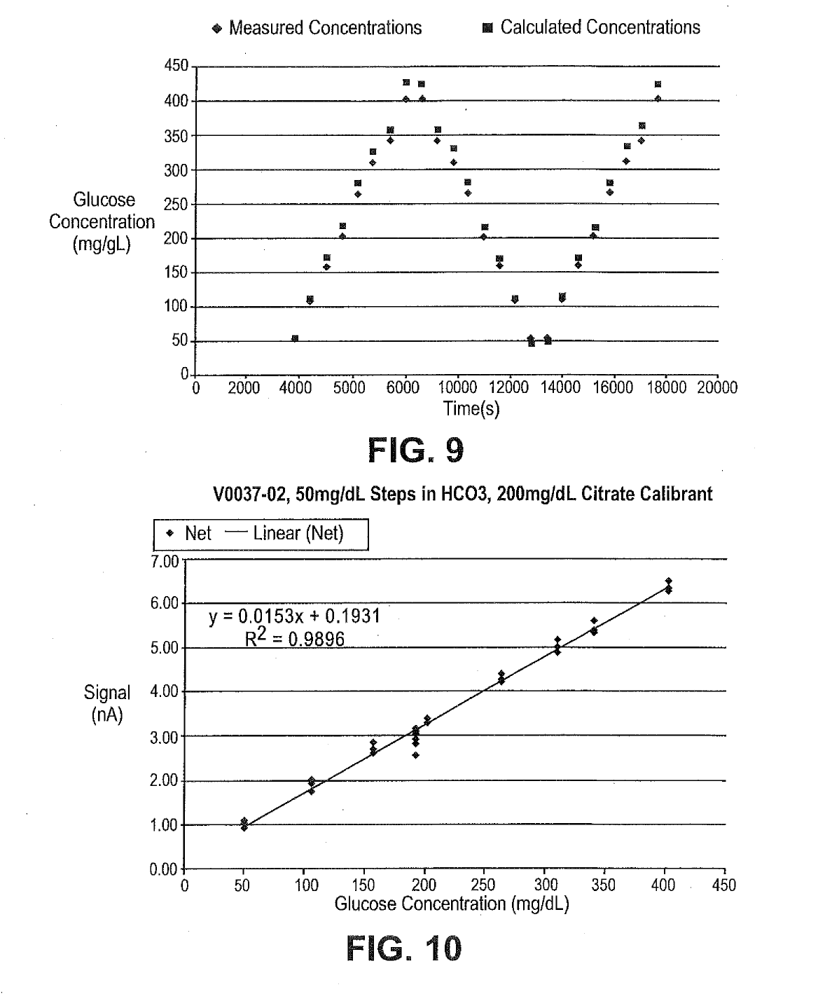

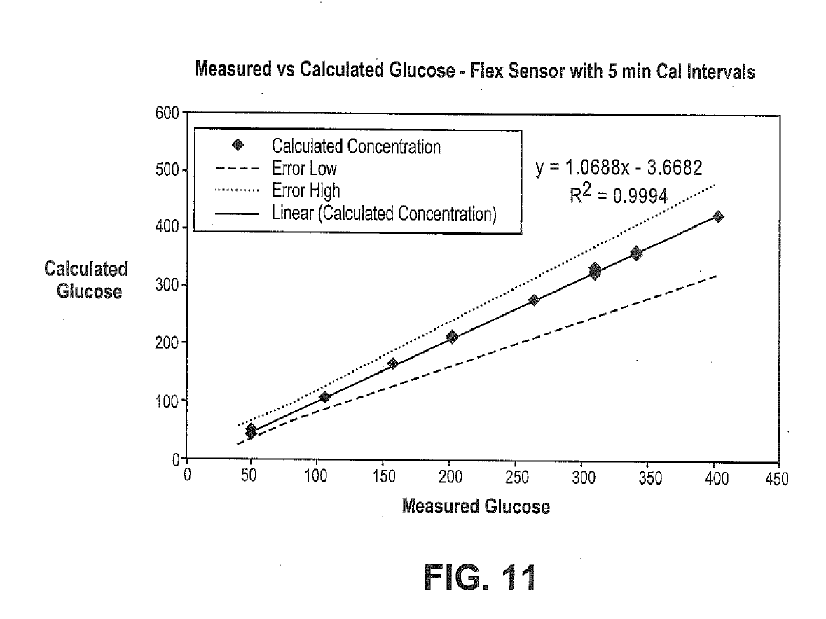

Calculated Concentrations vs. Measured Concentrations are shown in FIG. 9. As shown, the graph tracks the glucose concentration by the YSI and the calculated glucose concentrations. FIG. 10 shows the linearity of the first order fit of solutions. FIG. 11 shows First Calculated concentration versus the Measured Glucose from the YSI. The dotted lines of FIG. 11 indicate the error for this data set is ±15 mg/dL for concentrations from ≦40 to 75 mg/dL glucose and ±20% of the calculated glucose above 75 mg/dL per the current ISO specification for glucose analyzers for hospital use.

Thus, present embodiments provide for methods and systems for preparation and use of calibrant infusion fluid sources for intravenously positioned sensors. This method also provides a highly accurate sensor capable of preventing or eliminating thrombus for use in a hospital environment. In another embodiment, a highly accurate sensor capable of preventing or eliminating sensor drift resulting from an acidic environment about the enzyme is provided. In such embodiments, buffer agents provide physiological pH control during the calibration cycle.

While the foregoing disclosure discusses illustrative embodiments, it should be noted that various changes and modifications could be made herein without departing from the scope of the described aspects and/or embodiments as defined by the appended claims. Furthermore, although elements of the described aspects and/or embodiments may be described or claimed in the singular, the plural is contemplated unless limitation to the singular is explicitly stated. Additionally, all or a portion of any embodiment may be utilized with all or a portion of any other embodiment, unless stated otherwise.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other changes, combinations, omissions, modifications and substitutions, in addition to those set forth in the above paragraphs are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

What is claimed is:

1. A calibrant infusion fluid source comprising:
a container comprising a saline solution;
a predetermined amount of calibrant present in the saline solution;
an effective amount of at least one non-heparin, anti-thrombotic agent present in the saline solution;
wherein the calibrant infusion source is adaptable to a intravenous glucose sensor.

2. A calibrant infusion fluid source of claim 1, wherein the calibrant infusion fluid source further comprises a buffering system with sufficient buffering capacity such that a linear glucose verses current signal is obtained up to 1000 mg/dL glucose.

3. A calibrant infusion fluid source of claim 2, wherein the buffering system comprises bicarbonate ion between about 20 mM and about 100 mM such as to provide a physiological pH.

4. A calibrant infusion fluid source of claim 2, wherein the buffering system comprises phosphate ion between about 0.020 M and about 0.120 M such as to provide a physiological pH.

5. A calibrant infusion fluid source of claim 2, wherein the buffering system comprises bicarbonate ion and phosphate ion such as to provide a physiological pH.

6. A calibrant infusion fluid source of claim 2, wherein the pH of the infusion fluid source is between 6.50 and 7.6.

7. A calibrant infusion fluid source of claim 1, wherein the at least one non-heparin, anti-thrombotic agent is citrate, and the buffer system is selected from at least one of phosphate or bicarbonate, wherein the calibrant fluid source has an osmolality essentially the same as human blood.

8. A system for sensing an analyte of interest in a subject, the system comprising:
a calibrant infusion fluid source comprising:
a container comprising a saline solution;
a predetermined amount of calibrant present in the saline solution;
an amount of a non-heparin anti-thrombotic agent present in the saline solution sufficient to prevent or eliminate thrombus; and
a glucose sensor adapted for fluid communication with the calibrant infusion fluid source; and
a controller electrically coupled to the glucose sensor.

9. A system of claim 8, further comprises a buffering system with sufficient buffering capacity such that a linear glucose verses current signal is obtained up to 1000 mg/dL glucose.

10. A system of claim 9, wherein buffering system comprises bicarbonate ion between about 20 mM and about 100 mM such as to provide a physiological pH.

11. A system of claim 9, wherein buffering system comprises phosphate ion between about 0.020 M and about 0.120 M such as to provide a physiological pH.

12. A system of claim 9, wherein buffering system comprises bicarbonate ion and phosphate ion such as to provide a physiological pH.

13. A system of claim 9, wherein the pH of the infusion fluid source is between 6.50 and 7.6.

14. A system of claim 8, wherein the at least one non-heparin, anti-thrombotic agent is, citrate, and the buffer system is selected from at least one of citrate, phosphate, or bicarbonate, wherein the calibrant fluid source has an osmolality essentially the same as human blood.

15. A system of claim 8, further comprising a catheter adapted to house the glucose sensor.

16. A system of claim 15, wherein at least one of the surfaces of the catheter is surface treated to reduce or eliminate thrombus.

17. A system of claim 8, further comprising a housing adapted to receive the glucose sensor.

18. A system of claim 17, wherein at least one of the surfaces of the housing is surface treated to reduce or eliminate thrombus.

19. A method for preventing or eliminating thrombus during use of a sensor, the method comprising:
providing a calibrant infusion fluid source, the calibrant infusion fluid source comprising:
a saline solution;
a predetermined amount of calibrant present in the saline solution;
an amount of a non-heparin anti-thrombotic agent sufficient to prevent or eliminate thrombus present in the saline solution; and
presenting the calibrant infusion fluid to an intravenously implanted sensor, wherein at least a portion of the sensor is in contact with blood.

20. A method of claim 19, wherein the method further comprises providing a buffering system, optionally comprising citrate ion, with sufficient buffering capacity such that a linear glucose verses current signal is obtained up to 1000 mg/dL glucose.

21. A method of claim 20, wherein buffering system comprises bicarbonate ion between about 20 mM and about 100 mM such as to provide a physiological pH.

22. A method of claim 20, wherein buffering system comprises phosphate ion between about 0.020 M and about 0.120 M such as to provide a physiological pH.

23. A method of claim 20, wherein buffering system comprises bicarbonate ion and phosphate ion such as to provide a physiological pH.

24. A method of claim 20, wherein the pH of the infusion fluid source is between 6.50 and 7.6.

25. A method of claim 19, wherein the at least one non-heparin, anti-thrombotic agent is citrate, and the buffer system is selected from at least one of citrate, phosphate, or bicarbonate, wherein the calibrant fluid source has an osmolality essentially the same as human blood.

26. A method of claim 19, further comprising providing a catheter adapted to house the glucose sensor.

27. A method of claim 26, wherein at least one of the surfaces of the catheter is surface treated to reduce or eliminate thrombus.

28. A method of claim 19, further comprising a housing adapted to receive the glucose sensor.

29. A method of claim 28, wherein at least one of the surfaces of the housing is surface treated to reduce or eliminate thrombus.

30. A method of claim 19, further comprising maintaining a substantially constant pH environment about the glucose sensor during use.
